(19) United States
(12) Patent Application Publication
(10) Pub. No.: US 2023/0134702 A1
(43) Pub. Date: May 4, 2023

(54) FURNITURE CABINET WITH SHOE STERILIZATION CHAMBER

(71) Applicant: Lara Valdes, Park City, UT (US)

(72) Inventor: Lara Valdes, Park City, UT (US)

(21) Appl. No.: 17/960,717

(22) Filed: Oct. 5, 2022

Related U.S. Application Data
(60) Provisional application No. 63/233,681, filed on Aug. 16, 2021.

(51) Int. Cl. A61L 2/10; A61L 2/24; A61L 2/28; A47L 23/02

(52) U.S. Cl. CPC A61L 2/10; A61L 2/24; A61L 2/28; A47L 23/02; A61L 2202/11; A61L 2202/122; A61L 2202/14; A61L 2202/121

(57) ABSTRACT

A furniture cabinet with an internal UV sterilization system is provided to allow a person to sterilize shoes upon returning to a clean environment such as their residence.

### PRIORITY

The present application claims the benefit of U.S. Provisional Application Ser. No. 63/233,681, filed Aug. 16, 2021, which is herein incorporated by reference in its entirety.

### THE FIELD OF THE INVENTION

The present invention relates to a furniture cabinet useful for sterilizing shoes. In particular, examples of the present invention relate to a furniture cabinet with a UVC sterilization light that can be used to sterilize shoes to eliminate pathogens carried on the shoes.

### BACKGROUND

Shoes are routinely exposed to a number of pathogens such as bacteria and viruses as the shoes are worn. Locations such as streets, building corridors, and bathrooms typically have a large number of pathogens on them. These pathogens are picked up and carried on shoes and may ultimately be brought back to a person's residence or other locations. Shoes may be a common mode of contamination for flooring and other surfaces and may contribute to illness by carrying pathogens into living quarters.

### BRIEF DESCRIPTION OF THE DRAWINGS

Non-limiting and non-exhaustive examples of the present invention are described with reference to the following figures, wherein like reference numerals refer to like parts throughout the various views unless otherwise specified.

FIG. 1 is a drawing which shows a perspective view of a sterilization furniture cabinet.

FIG. 2 is a drawing which shows a front view of the sterilization furniture cabinet.

FIG. 3 is a drawing which shows a side view of the sterilization furniture cabinet.

FIG. 4 is a drawing which perspective view of the sterilization furniture cabinet with the front door panel open.

FIG. 5 is another drawing which perspective view of the sterilization furniture cabinet with the front door panel open.

FIG. 6 is a schematic drawing which shows electrical components of the sterilization furniture cabinet.

Corresponding reference characters indicate corresponding components throughout the several views of the drawings. Unless otherwise noted, the drawings have been drawn to scale. Skilled artisans will appreciate that elements in the figures are illustrated for simplicity and clarity. For example, the dimensions of some of the elements in the figures may be exaggerated relative to other elements to help improve understanding of various examples of the present invention. Also, common but well-understood elements that are useful or necessary in a commercially feasible embodiment are often not depicted in order to facilitate a less obstructed view of these various embodiments of the present invention.

It will be appreciated that the drawings are illustrative and not limiting of the scope of the invention which is defined by the appended claims. The examples shown each accomplish various different advantages. It is appreciated that it is not possible to clearly show each element or advantage in a single figure, and as such, multiple figures are presented to separately illustrate the various details of the examples in greater clarity. Similarly, not every example need accomplish all advantages of the present disclosure.

### DETAILED DESCRIPTION

In the following description, numerous specific details are set forth in order to provide a thorough understanding of the present invention. It will be apparent, however, to one having ordinary skill in the art that the specific detail need not be employed to practice the present invention. In other instances, well-known materials or methods have not been described in detail in order to avoid obscuring the present invention.

In the above disclosure, reference has been made to the accompanying drawings, which form a part hereof, and in which are shown by way of illustration specific implementations in which the disclosure may be practiced. It is understood that other implementations may be utilized and structural changes may be made without departing from the scope of the present disclosure. References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, such feature, structure, or characteristic may be used in connection with other embodiments whether or not explicitly described. The particular features, structures or characteristics may be combined in any suitable combination and/or sub-combinations in one or more embodiments or examples. It is appreciated that the figures provided herewith are for explanation purposes to persons ordinarily skilled in the art.

Implementations of the systems, devices, and methods disclosed herein may comprise or utilize a special purpose or general-purpose computer including computer hardware, such as, for example, one or more processors and system memory, as discussed herein. Implementations within the scope of the present disclosure may also include physical and other computer-readable media for carrying or storing computer-executable instructions and/or data structures. Such computer-readable media can be any available media that can be accessed by a general purpose or special purpose computer system to store or transmit computer-executable instructions or data.

As used herein, “adjacent” refers to near or close sufficient to achieve a desired effect. Although direct contact is common, adjacent can broadly allow for spaced apart features.

As used herein, the singular forms “a,” and, “the” include plural referents unless the context clearly dictates otherwise.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be such as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a number or numerical range endpoint by providing that a given value may be one above or one below the number or endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Dimensions, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

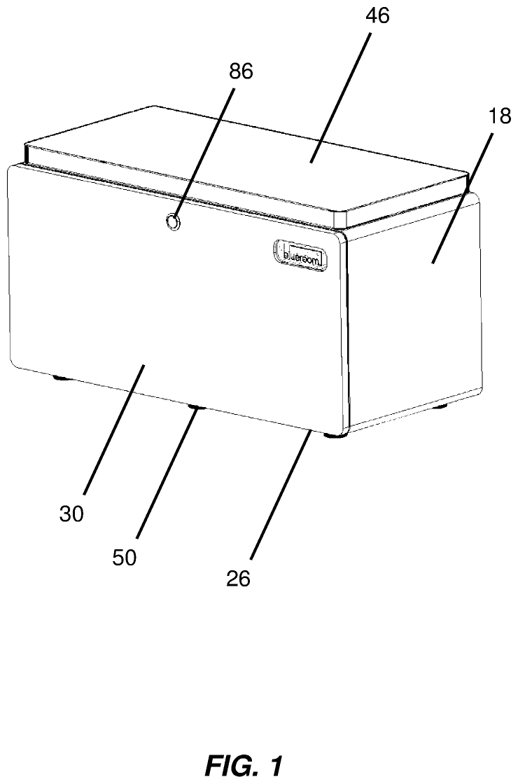

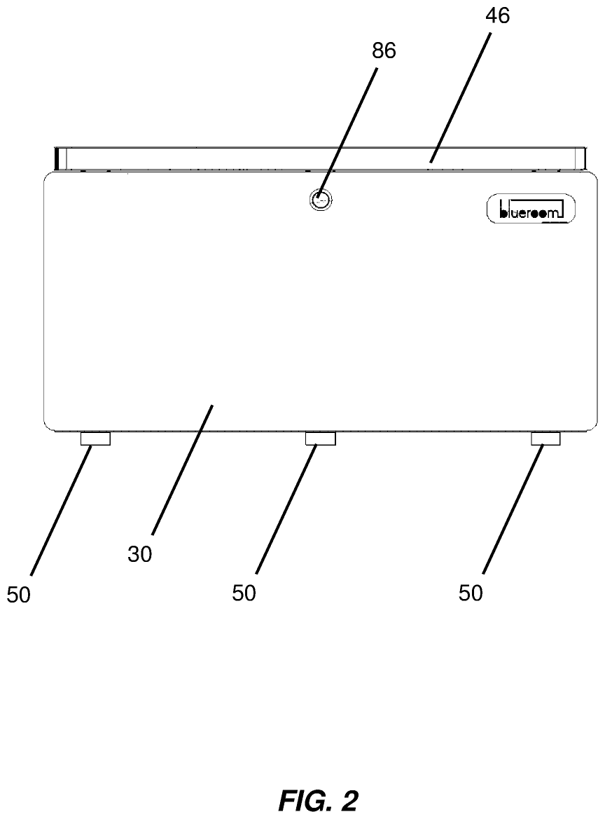

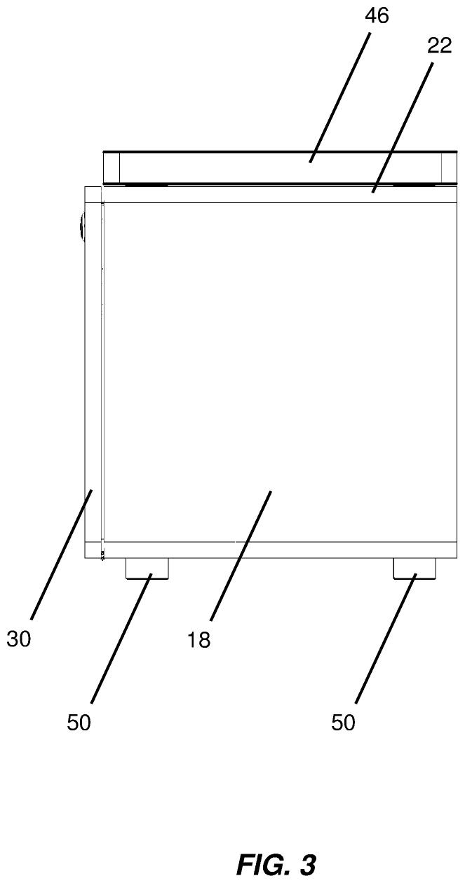

Turning now to FIGS. 1 through 5, a sterilization furniture cabinet 10 is shown. The sterilization furniture cabinet 10 includes a wood cabinet which forms the structural body of the device. As discussed herein, a furniture cabinet refers to a piece of furniture which includes a cabinet, cabinet enclosure, or a similar enclosed cabinet space. The furniture cabinet includes a door to provide access to the cabinet space and to restrict access to the cabinet space during operation of the device. For example, the furniture cabinet may be a bench with a cabinet as part of the bench. The bench may have a simple design which blends with modern office furniture or may have a more ornamental design which complements home furnishings. The furniture cabinet may be designed to be free standing or alternatively to be attached to a wall or built in as part of the building structure.

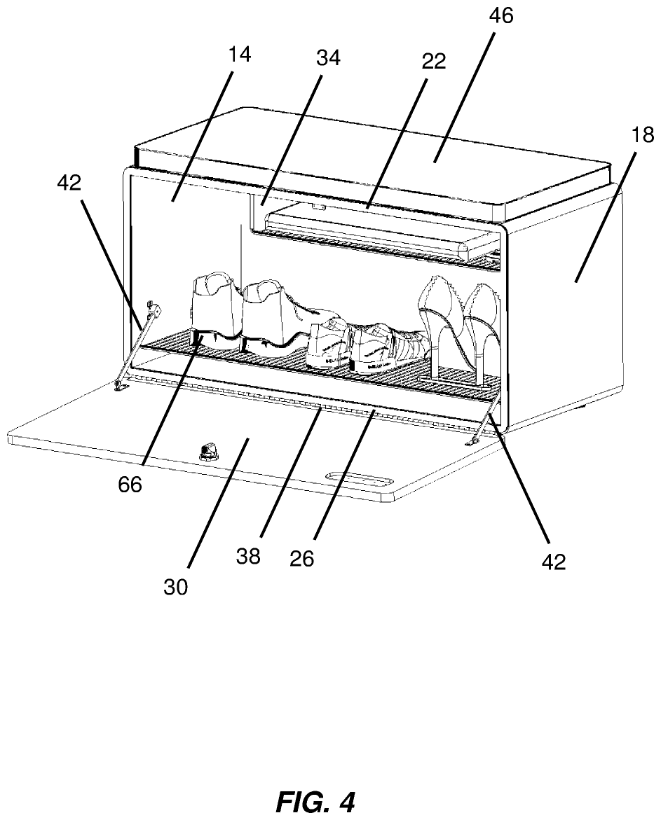

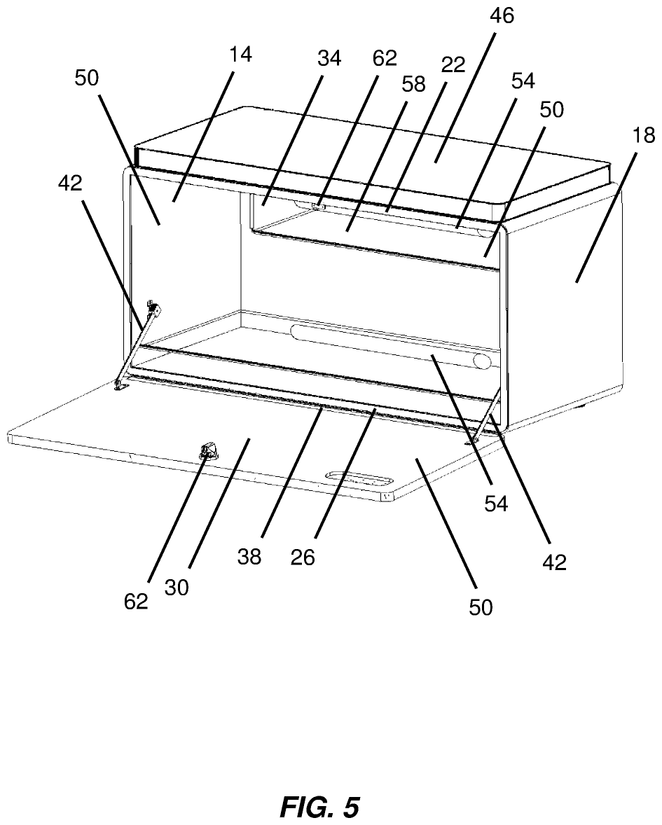

The cabinet 10 includes a left side panel 14, a right side panel 18, a top panel 22, a bottom panel 26, a front panel 30, and a back panel 34. The left panel 14, right panel 18, top panel 22, bottom panel 26, and back panel 34 are attached together and form a rigid and durable furniture cabinet with a front opening. The front panel 30 is attached to the furniture cabinet with one or more hinges 38 and can swing open or closed to permit access to the interior of the furniture cabinet 10. The front panel 30 may also be attached to the furniture cabinet 10 with door stops 42 which limit how far the front panel 30 is allowed to swing open. The door stops 42 are particularly useful when the front panel 30 swings downwardly to open the cabinet as shown in this example. FIGS. 4 and 5 show the front panel in an open position.

The example panels of the furniture cabinet 10 are constructed of wood and provide an aesthetically pleasing article of furniture that may function as a bench and may be placed in a building entry area or other location. The furniture cabinet 10 may include a cushion 46 which is attached to the top panel 22 if the furniture cabinet 10 is used as a bench. The example furniture cabinet 10 is greater than 1 foot wide, greater than 1 foot tall, and greater than 1 foot deep front to back. This example is approximately two or three feet wide, approximately one and a half feet tall, and approximately one and a half feet deep front to back. The furniture cabinet 10 is of sufficient size to receive at least a pair of shoes therein, and the example cabinet 10 is of sufficient size to receive several pair of shoes therein.

The example front panel 30 forms the cabinet door. In the example configuration, a single door panel 30 is used and the door provides access to the entire interior space within the furniture cabinet 10.

FIGS. 4 and 5 show the front door panel 30 open and show the interior of the furniture cabinet 10. The interior sides of the left side panel 14, right side panel 18, top panel 22, bottom panel 26, front panel 30, and back panel 34 are faced with mirrored surfaces 50. In the example furniture cabinet 10, the interior sides of these panels are covered with a mirror polished aluminum sheet to provide the mirrored interior surfaces 50. The mirrored interior surfaces may comprise polished metal surfaces or mirrored glass or mirrored acrylic surfaces. One or more UV producing germicidal bulbs 54 is mounted to the interior of the furniture cabinet 10. In the example furniture cabinet 10, the example germicidal bulbs 54 are G25T8 UV producing germicidal bulbs. The furniture cabinet 10 includes ballasts or power supplies to operate the bulbs as needed. In the example furniture cabinet, two germicidal bulbs 54 are used and these bulbs 54 are mounted laterally adjacent the top and bottom of the back panel 34.

The furniture cabinet 10 includes one or more shelves 58. The example furniture cabinet 10 includes a first shelf mounted near the bottom of the cabinet 10 and a second shelf mounted near the top of the cabinet. The shelves 54 may be made from a material which transmits UV light. The example shelves are made from UV transmissive acrylic. Alternatively, the shelves 58 could be made from wire rack shelving as this has a high degree of open space and a high surface reflectivity.

In the example furniture cabinet, the UV lights 54 are placed above the top shelf 58 and below the bottom shelf 58. This provides a high degree of UV light beneath the bottom shelf while also generally bathing the entire interior of the furniture cabinet in UV light even while objects are placed onto the shelves 58. This is in part due to the UV reflective mirrored surfaces 50 lining the interior of the furniture cabinet 10. The light from the UV bulbs 54 is reflected from the cabinet interior surfaces and allowed to pass through the shelves 58, bathing objects inside of the cabinet 10 in UV light.

The furniture cabinet 10 includes a latch 62 which may include a door latch portion and a cabinet body latch portion. The latch 62 holds the door 30 in a closed position and may protect against the door 30 being inadvertently opened while the UV germicidal bulbs 54 are operating. The latch 62 or an open/closed interlock switch may be electrically connected to the UV light controller to only allow the germicidal bulbs 54 to operate while the door 30 is closed.

FIG. 4 shows how objects, shoes 66 in particular, may be placed onto the shelves 58 to be sterilized. While the furniture cabinet is operating, the shoes 66 are surrounded in UV light and germs on the shoes are killed. The UV light reaches the interior of the shoes 66 as well as the bottom of the shoes 66. Using the sterilization furniture cabinet 10 at the entrance to a residential space or other location will keep many germs from being tracked into the location on shoes. People may, on arrival at the location, remove their shoes and place them into the sterilization furniture cabinet 10 to sterilize the shoes.

Changing the style of the cabinet 10 allows the sterilization furniture cabinet to be used as different types of furniture. In one example, the furniture cabinet may be styled as a bench. The top of the cabinet 10 may include a cushion 46 which makes the cabinet more comfortable for persons to sit on. If desired, the top panel of the cabinet 10 may form the door; facilitating access to the interior of the cabinet while the cabinet is on the floor. The furniture cabinet 10 may be made in a variety of decorative or ornate styles to match different decorating tastes. This cabinet is useful in many home and office locations to allow persons to sterilize their shoes upon arrival.

In another example, the furniture cabinet may be styled in a more contemporary style and may be mounted on a wall, as part of a bookcase, or even as part of modular cubical furniture and may be mounted near an office entrance or workspace entrance. In this example, the front panel of the cabinet will typically form the door. The sterilization furniture cabinet blends into the office decor and allows workers or guests to sterilize their shoes upon arrival.

In another example, the furniture cabinet may be configured to be built into a building and allow for customization. For example, the cabinet may be installed into a built-in bench, bookcase, wall, or entry nook as an integrated part of the building. The top, bottom, front, back, and sides of the cabinet may be materials such as plywood which facilitate permanent assembly into the building structure. The cabinet may be partially assembled and the exterior of the cabinet may be unfinished as it would typically be concealed within the building once installed and exposed portions of the cabinet would be finished to match the building construction. The door may be configured to receive a supplemental door panel such as a cabinet door panel so that the sterilization furniture cabinet matches surrounding cabinets or building features. This allows the sterilization furniture cabinet to be permanently installed as part of a building such as a custom home and allows the cabinet to blend seamlessly with the desired home style.

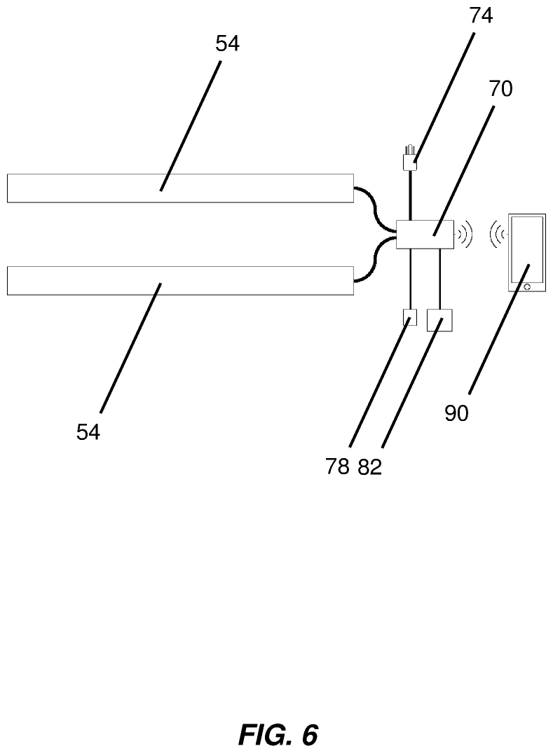

FIG. 6 shows a schematic drawing of the electrical/electronic components of the furniture cabinet 10. The cabinet 10 includes an electrical controller 70. The controller 70 is electrically connected to the sterilization bulbs 54 and may include a ballast or driver or connect to the bulbs via a ballast or driver to operate the bulbs 54 depending on the requirements of the particular bulbs 54. The controller 70 includes a power source or connector 74 to connect to a power source such as building mains power which provides power to the furniture cabinet 10. The controller 70 is electrically connected to a door safety switch 78. The door safety switch may be part of the latch 62 or a door interlock switch as discussed above. The controller 70 senses whether the door is open or closed via the door safety switch 78 and does not operate the bulbs 54 if the door 30 is open. The controller 70 is also connected to a user interface 82 which allows a user to control operation of the furniture cabinet 10. The user interface 82 may be a simple on/off button such as button 86 on the front of the door 30. Button 86 may alternatively be a mechanical button to open or close the door 30 and a user interface may be located on another area of the cabinet 10. The user interface 82 primarily allows a user to turn the sterilization lamps 54 on or off and operate the sterilization cabinet 10. The user interface may be a control panel which also displays the elapsed or remaining sterilization time and which allows for additional operational control over the cabinet 10. The user interface may also include an indicator which indicates the status of the sterilization cabinet 10. For example, the user interface may display a light in a first color if the sterilization cabinet 10 has completed a timed sterilization cycle without interruption and may display a light in a second color if the sterilization cabinet 10 was interrupted during a sterilization cycle. Alternately, the user interface may display a light in a first color if the sterilization bulbs 54 are on and a second color if the sterilization cabinet has completed a sterilization cycle and the bulbs 54 are off.

In another example, the controller 70 may communicate wirelessly with a remote control panel 90 such as a cell phone 90 or tablet computer 90. The controller 70 may include a wireless transmitter/receiver which communicates through Wi-Fi or Bluetooth. The control panel 90 may allow a user to start or stop a sterilization cycle in the furniture cabinet 10 as well as to monitor the sterilization cycle. The user interface 82 and/or the remote control panel 90 may display sterilization cycle length, the elapsed sterilization time, and the remaining sterilization time. The user interface 82 and/or the remote control panel 90 may also alert the user and display if the sterilization cycle was interrupted and may also alert the user and display to the user when a sterilization cycle has completed.

The controller 70 typically includes input and output communications, a timer, and a processor which is configured and programmed to implement the functionality discussed above. In a simple form, the controller 70 may receive a start input and operate the sterilization bulbs 54 for a predetermined period of time and then shut off the sterilization bulbs.

A sterilization cycle will typically last a predetermined duration of time. When a sterilization cycle is initiated, the controller 70 may verify the status of the door safety switch 78, operate the sterilization lamps 54 if the door safety switch 78 indicates that the door is closed, and monitor the elapsed time of the sterilization cycle typically through a count-down timer. During the sterilization cycle the controller 70 will continue to monitor the door safety switch 78. If the door safety switch 78 indicates that the door 30 is opened during the sterilization cycle, the lamps 54 are turned off and the user is alerted that the sterilization cycle has been interrupted and is incomplete. If the controller 70 detects, via the door safety switch 78, that the door 30 remains closed during the sterilization cycle, the controller 70 will continue to operate the UV emitters/sterilization lamps 54 throughout the sterilization cycle and will then indicate to the user that the cleaning cycle is completed.

The sterilization cabinet 10 is particularly useful in sterilizing objects such as shoes which accumulate a large number of germs when in use and which are desirable to sterilize when returning to a clean environment such as a living space.

The above description of illustrated examples of the present invention, including what is described in the Abstract, is not intended to be exhaustive or to be limiting to the precise forms disclosed. While specific examples of the invention are described herein for illustrative purposes, various equivalent modifications are possible without departing from the broader scope of the present claims. Indeed, it is appreciated that specific example dimensions, materials, voltages, currents, frequencies, power range values, times, etc., are provided for explanation purposes and that other values may also be employed in other examples in accordance with the teachings of the present invention.

What is claimed is:

1. A UV sterilization furniture cabinet comprising:
a cabinet defining an internal enclosed volume;
a door which is movable between a first closed position and a second open position to selectively allow access to the internal enclosed volume;
an ultraviolet emitter disposed in the internal enclosed volume;
an electrical controller electrically connected to the ultraviolet emitter to selectively operate the ultraviolet emitter;
a shelf which passes ultraviolet light therethrough disposed in the internal enclosed volume; and
wherein the shelf is configured to receive a pair of shoes thereon such that the shoes are spaced apart from an internal surface of the cabinet.

2. The furniture cabinet of claim 1, wherein the cabinet comprises an upper cabinet horizontal exterior surface which is sized and configured for allowing a person to sit thereon.

3. The furniture cabinet of claim 2, further comprising a seat cushion disposed on the upper cabinet horizontal exterior surface.

4. The furniture cabinet of claim 1, wherein the internal surface of the cabinet comprises a material which reflects ultraviolet light.

5. The furniture cabinet of claim 1, further comprising a door safety switch connected to the door and electrically connected to the controller whereby the controller is configured to operate the ultraviolet emitter only if the door is closed.

6. The furniture cabinet of claim 1, further comprising a user interface configured to allow a user to start operation of the cabinet to thereby operate the electrical controller and ultraviolet emitter to sterilize the shoes during a sterilization cycle.

7. The furniture cabinet of claim 6, wherein the user interface is configured to indicate to the user if the sterilization cycle has completed successfully.

8. The furniture cabinet of claim 6, wherein the user interface is configured to indicate to the user if the sterilization cycle has been interrupted.

9. The furniture cabinet of claim 1, wherein the cabinet is installed as a built in cabinet in a home.

10. The furniture cabinet of claim 1, wherein the cabinet is greater than 1 foot wide, greater than 1 foot tall, and greater than 1 foot deep front to back.

11. The furniture cabinet of claim 1, wherein the cabinet is installed in a building near an entrance.

12. A UV sterilization furniture cabinet comprising:
an internal enclosed volume;
a door which is movable between a first closed position and a second open position to selectively allow access to the internal enclosed volume;
an ultraviolet emitter disposed in the internal enclosed volume;
an electrical controller electrically connected to the ultraviolet emitter to selectively operate the ultraviolet emitter; and
wherein the internal enclosed volume is of sufficient size to receive a pair of shoes therein.

13. The furniture cabinet of claim 12, further comprising a shelf which passes ultraviolet light therethrough disposed in the internal enclosed volume, and wherein the shelf is configured to receive a pair of shoes thereon such that the shoes are spaced apart from an internal surface of the cabinet.

14. The furniture cabinet of claim 12, further comprising a cabinet having a front, a back, a first side, a second side, a bottom, and a top, and wherein the cabinet defines the internal enclosed volume.

15. The furniture cabinet of claim 12, wherein the cabinet comprises an upper cabinet horizontal exterior surface which is sized and configured for allowing a person to sit thereon.

16. The furniture cabinet of claim 15, further comprising a seat cushion disposed on the upper cabinet horizontal exterior surface.

17. The furniture cabinet of claim 12, wherein the internal surface of the cabinet comprises a material which reflects ultraviolet light.

18. The furniture cabinet of claim 12, wherein the cabinet is installed as a built in cabinet in a home.

19. The furniture cabinet of claim 12, wherein the cabinet is greater than 1 foot wide, greater than 1 foot tall, and greater than 1 foot deep front to back.

20. The furniture cabinet of claim 12, wherein the cabinet is installed in a building near an entrance.
